(19) United States
(12) Patent Application Publication
(10) Pub. No.: US 2023/0279011 A1
(43) Pub. Date: Sep. 7, 2023

(54) WATER-SOLUBLE TRYPTHANTRIN DERIVATIVES FOR REDOX FLOW BATTERIES

(71) Applicant: UNIVERSIDADE DE COIMBRA, Coimbra (PT)

(72) Inventors: Marta PIÑEIRO GÓMEZ, Coimbra (PT); Daniela RIBEIRO PINHEIRO, Coimbra (PT); João Sérgio SEIXAS DE MELO, Coimbra (PT)

(21) Appl. No.: 18/026,275

(22) PCT Filed: Sep. 9, 2021
(86) PCT No.: PCT/IB2021/058195

§ 371 Date: Mar. 14, 2023

(30) Foreign Application Priority Data
Sep. 11, 2020 (PT) 116722

(51) Int. Cl. C07D 487/04; H01M 8/18

(52) U.S. Cl. CPC C07D 487/04; H01M 8/188

(57) ABSTRACT

The present disclosure relates to a new classes of water-soluble trypthantrin derivatives of Formula (I) and its salts or Formula (II) and its salts, and their use as soluble electrolytes (active materials) for aqueous organometallic and all-organic redox flow batteries (RFB) working at neutral pH with long-term stability.

Electrochemical measurements show that water soluble trypthantrin derivatives display reversible peaks at several pH values, allowing its use as the anolyte together with organometallic and organic water-soluble catholytes in a neutral supporting electrolyte. The single cell tests show reproducible charge-discharge cycles for both type of catholytes with significant improvement results for the aqueous all-organic RFB, with coulombic (89%), voltaic (75%) and energetic (67%) efficiencies stabilized during 50 working cycles

### TECHNICAL FIELD

The present disclosure relates to a new class of water-soluble trypthantrin derivatives and its salts and their use as components of redox flow batteries. In particular, this disclosure provides the identification and characterization of sulfonic acid and amine derivatives of trypthantrin, as well as their salts, with redox properties adequate for their use as electrolytes in inorganic/organic or all organic aqueous redox flow batteries, which proved to be highly efficient, with reproducible charge-discharge cycles, and efficiencies, stabilized during at least 250 working cycles.

### BACKGROUND ART

The global and economic population growth impels the increase of energy consumption. Resources formed over hundreds of millions of years have been burned in a relatively short time, with substantial environmental impact (Alotto, P. et al. Redox flow batteries for the storage of renewable energy: a review. Renew. Sustain. Energy Rev. 29, 325-335 (2014) and Carretero-González, J. et al. Highly water-soluble three-redox state organic dyes as bifunctional analytes. Energy Environ. Sci. 9, 3521-3530 (2016)). To reduce the use of fossil fuels, and environmentally friendly route to generate and store electricity from renewable sources is needed to fulfill the world's needs in a sustainable way (Khalid, A. & Awad, M. Chapter 5—Redox—Principles and Advanced Applications. (IntchOpen, 2017); Huskinson, B. et al. A metal-free organic-inorganic aqueous flow battery. Nature 505, 195 (2014); Liu, W. et al. Aqueous flow batteries: research and development. Chem. Eur. J. 25, 1649-1664 (2019); Shigematsu, T. Redox flow battery for energy storage. Sei Tech. Rev. 73, 4-13 (2011) and Noack, J. et al. The chemistry of redox-flow batteries. Angew. Chem. Int. Ed. 54, 9776-9809 (2015)).

In the past recent years, renewable energy technologies have therefore attracted much scientific and public interest, as disclosed in Wei, X. et al. Materials and systems for organic redox flow batteries: status and challenges. ACS Energy Lett. 2, 2187-2204 (2017). Traditional batteries such as lithium-ion batteries are of widespread use, but they cannot cost-effectively store, enough energy for the long discharge durations at rated power, they use flammable organic electrolytes and reveal high maintenance costs (Singh, V. et al. R. Aqueous organic redox flow batteries. Nano Res. 12, 1988-2001 (2019) and Winsberg, J. et al. Redox-flow batteries: from metals to organic redox-active materials. Ang. Chem. Int. Ed. Eng. 56, 686-711 (2017)).

Redox flow batteries (RFBs) are an emerging and highly promising power source, representing one of the best storage technologies for electrical energy that is obtained from renewable sources like wind power and solar energy, as disclosed in Winsberg et al, TEMPO/Phenazine Combi-Molecule: A Redox-Active Material for Symmetric Aqueous Redox-Flow Batteries. ACS Energy Letters 2016, 1 (5), 976-980.

RFBs are frequently described as affordable, reliable (with extremely long charge/discharge cycle life) and eco-friendly depending on the materials used, according to several fonts, for example: (a) Huang et al, N,N′-Disubstituted Indigos as Readily Available Red-Light Photoswitches with Tunable Thermal Half-Lives. Journal of the American Chemical Society 2017, 139 (42), 15205-15211; (b) Lin et al, Alkaline Quinone Flow Battery. Science 2015, 349 (6255), 1529-1532; (c) Wang et al, Recent Progress in Redox Flow Battery Research and Development. Advanced Functional Materials 2013, 23 (8), 970-986; (d) Chen, R., Redox Flow Batteries for Energy Storage: Recent Advances in Using Organic Active Materials. Current Opinion in Electrochemistry 2020, 21, 40-45; (e) Liu et al, A Sustainable Redox Flow Battery with Alizarin-Based Aqueous Organic Electrolyte. ACS Applied Energy Materials 2019, 2 (4), 2469-2474.

Aqueous organic redox flow batteries (AORFBs) have been recently proposed as low-cost and alternatives to the metal-based RFBs technology, as revealed in Liu et al, A Sustainable Redox Flow Battery with Alizarin-Based Aqueous Organic Electrolyte. ACS Applied Energy Materials 2019, 2 (4), 2469-2474; and in Singh et al, Aqueous Organic Redox Flow Batteries. Nano Research 2019, 12, 1988-2001; (b) Liu et al, Aqueous Flow Batteries: Research and Development. Chemistry—A European Journal 2019, 25 (7), 1649-1664; (c) Hoober-Burkhardt et al, A New Michael-Reaction-Resistant Benzoquinone for Aqueous Organic Redox Flow Batteries. Journal of The Electrochemical Society 2017, 164 (4), A600-A607; (d) Winsberg et al, Redox-Flow Batteries: From Metals to Organic Redox-Active Materials. Angewandte Chemie International Edition 2017, 56 (3), 686-711; (e) Wei et al, Materials and Systems for Organic Redox Flow Batteries: Status and Challenges. ACS Energy Letters 2017, 2 (9), 2187-2204; (f) Lv et al, Structure Reorganization-Controlled Electron Transfer of Bipyridine Derivatives as Organic Redox Couples. Journal of Materials Chemistry A 2019, 7 (47), 27016-27022; (g) Luo et al, Unprecedented Capacity and Stability of Ammonium Ferrocyanide Catholyte in pH Neutral Aqueous Redox Flow Batteries. Joule 2019, 3 (1), 149-163.

These AORFBs have several outstanding advantages mainly because of their prospect of offering such a grid-scale energy storage solution, as commented in Hu et al, Long-Cycling Aqueous Organic Redox Flow Battery (AORFB) toward Sustainable and Safe Energy Storage. Journal of the American Chemical Society 2017, 139 (3), 1207-1214. In addition, AORFBs are more environmentally friendly and safe since they use non-flammable aqueous redox-active electrolytes, as presented in Singh et al, Aqueous Organic Redox Flow Batteries. Nano Research 2019, 12, 1988-2001; and in DeBruler et al, Designer Two-Electron Storage Viologen Anolyte Materials for Neutral Aqueous Organic Redox Flow Batteries. Chem 2017, 3 (6), 961-978.

### SUMMARY OF INVENTION

The present disclosure relates to trypthantrin derivatives with the general formula (I) and its salts, and trypthantrin derivatives with general formula (II) and its salts. Formula (I) represent trypthantrin derivatives with H, halogen, alkyl, or aryl groups at the aromatic ring and at least one sulfonic acid substituent. Formula (II) represent trypthantrin derivatives with H, halogen, alkyl, or aryl groups at the aromatic ring and at least one amine group.

The present disclosure relates the assembly of aqueous redox flow batteries using compounds with general formula (I) or general formula (II), as well as its salts, as electrolyte.

### Technical Problem

In the last years, novel AORFBs designs have been proposed essentially based on different electrolytes, as it is revealed in Singh et al, Aqueous Organic Redox Flow Batteries. Nano Research 2019, 12, 1988-2001; in Winsberg et al, Redox-Flow Batteries: From Metals to Organic Redox-Active Materials. Angewandte Chemie International Edition 2017, 56 (3), 686-711; in Wei et al, Materials and Systems for Organic Redox Flow Batteries: Status and Challenges. ACS Energy Letters 2017, 2 (9), 2187-2204; in Hofmann, J. D.; Schroder, D., Which Parameter is Governing for Aqueous Redox Flow Batteries with Organic Active Material? Chemie Ingenieur Technik 2019, 91 (6), 786-794; and in Hollas et al, A Biomimetic High-Capacity Phenazine-Based Anolyte for Aqueous Organic Redox Flow Batteries. Nature Energy 2018, 3 (6), 508-514. Use of anthraquinones has gained popularity since these quinones are well known redox active molecules with electrochemical reversibility and fast reactions rates, according to Singh et al, Aqueous Organic Redox Flow Batteries. Nano Research 2019, 12, 1988-2001; Liu et al, Aqueous Flow Batteries: Research and Development. Chemistry—A European Journal 2019, 25 (7), 1649-1664; Lee et al, Performance evaluation of aqueous organic redox flow battery using anthraquinone-2,7-disulfonic acid disodium salt and potassium iodide redox couple. Chemical Engineering Journal 2019, 358, 1438-1445; Lee et al, Neutral pH Aqueous Redox Flow Batteries Using an Anthraquinone-Ferrocyanide Redox Couple. Journal of Materials Chemistry C 2020; Permatasari et al, Performance Improvement by Novel Activation Process Effect of Aqueous Organic Redox Flow Battery using Tiron and Anthraquinone-2,7-Disulfonic Acid Redox Couple. Chemical Engineering Journal 2020, 383, 123085; and Gerken et al, Comparison of Quinone-Based Catholytes for Aqueous Redox Flow Batteries and Demonstration of Long-Term Stability with Tetrasubstituted Quinones. Advanced Energy Materials 2020, 10 (20), 2000340. However, and in particular at alkaline media, this family of compounds still seems rather unstable and most of these quinone-based RFBs cannot meet requirements for practical application, as pointed out in Wei et al, Materials and Systems for Organic Redox Flow Batteries: Status and Challenges. ACS Energy Letters 2017, 2 (9), 2187-2204. Therefore, the search and development of new water-soluble electrolytes for improvement of battery storage systems is increasingly significant and will continue to grow in the future.

In recent years, the development of neutral pH AORFBs has stood out as promising RFBs technology for sustainable and safe energy storage, as presented in Singh et al, Aqueous Organic Redox Flow Batteries. Nano Research 2019, 12, 1988-2001; in Hu et al, A pH-Neutral, Metal-Free Aqueous Organic Redox Flow Battery Employing an Ammonium Anthraquinone Anolyte. Angewandte Chemie International Edition 2019, 58 (46), 16629-16636; in Beh et al, A Neutral pH Aqueous Organic-Organometallic Redox Flow Battery with Extremely High-Capacity Retention. ACS Energy Letters 2017, 2 (3), 639-644; and in Hu et al, Boosting the energy efficiency and power performance of neutral aqueous organic redox flow batteries. Journal of Materials Chemistry A 2017, 5 (42), 22137-22145. One of the reasons is the fact that, neutral pH-based electrolytes are less-corrosive, as revealed in Luo et al, Unprecedented Capacity and Stability of Ammonium Ferrocyanide Catholyte in pH Neutral Aqueous Redox Flow Batteries. Joule 2019, 3 (1), 149-163; in Hu et al, Long-Cycling Aqueous Organic Redox Flow Battery (AORFB) toward Sustainable and Safe Energy Storage. Journal of the American Chemical Society 2017, 139 (3), 1207-1214; in Hu et al, A pH-Neutral, Metal-Free Aqueous Organic Redox Flow Battery Employing an Ammonium Anthraquinone Anolyte. Angewandte Chemie International Edition 2019, 58 (46), 16629-16636; in Beh et al; Gordon, R. G.; Aziz, M. J., A Neutral pH Aqueous Organic-Organometallic Redox Flow Battery with Extremely High-Capacity Retention. ACS Energy Letters 2017, 2 (3), 639-644; in Hu et al, Boosting the energy efficiency and power performance of neutral aqueous organic redox flow batteries. Journal of Materials Chemistry A 2017, 5 (42), 22137-22145; and in Chen et al, Recent Progress in Organic Redox Flow Batteries: Active Materials, Electrolytes and Membranes. Journal of Energy Chemistry 2018, 27 (5), 1304-1325. Another reason is the fact that, neutral pH-based electrolytes are more eco-friendly when compared with acidic or alkaline electrolytes that suffer from corrosion problems in cell stacks, as presented in Lin et al, Alkaline Quinone Flow Battery. Science 2015, 349 (6255), 1529-1532; in Huskinson et al, A Metal-Free Organic-Inorganic Aqueous Flow Battery. Nature 2014, 505, 195; and in Lin et al, A Redox-Flow Battery with an Alloxazine-Based Organic Electrolyte. Nature Energy 2016, 1 (9), 16102.

Tryptanthrin and its derivatives are a surprising family of compounds with biological and pharmacological activities, as commented in Kaur et al, Recent Synthetic and Medicinal Perspectives of Tryptanthrin. Bioorganic & Medicinal Chemistry 2017, 25 (17), 4533-4552; in Deryabin et al, Synthesis and antimicrobial activity of tryptanthrin adducts with ketones. Russian Journal of Organic Chemistry 2017, 53 (3), 418-422; in Novak et al, Scanning Tunneling Microscopy of Indolo[2,1-b]quinazolin-6,12-dione (tryptanthrin) on HOPG: Evidence of Adsorption-Induced Stereoisomerization. Surface Science 600 2006, (20), L269-L273; in Bhattacharjee et al, Analysis of Stereoelectronic Properties, Mechanism of Action and Pharmacophore of Synthetic Indolo[2,1-b]quinazoline-6,12-dione Derivatives in Relation to Antileishmanial Activity Using Quantum Chemical, Cyclic Voltammetry and 3-D-QSAR CATALYST Procedures. Bioorganic & Medicinal Chemistry 2002, 10 (6), 1979-1989; in Kawakami et al, Antibacterial and Antifungal Activities of Tryptanthrin Derivatives. Transactions of the Materials Research Society of Japan 2011, 36 (4), 603-606; in Filatov et al, Concise Synthesis of Tryptanthrin Spiro Analogues with In Vitro Antitumor Activity Based on One-Pot, Three-Component 1,3-Dipolar Cycloaddition of Azomethine Ylides to Cyclopropenes. Synthesis 2019, 51 (03), 713-729; and in Amara et al, Conversion of Isatins to Tryptanthrins, Heterocycles Endowed with a Myriad of Bioactivities. European Journal of Organic Chemistry 2019, (31-32), 5302-5312.

Tryptanthrin and its derivatives also have the additional feature of displaying interesting redox properties due to the electron-accepting ability of the tryptanthrin structure, as presented in Klimovich et al, A comparative assessment of the effects of alkaloid tryptanthrin, rosmarinic acid, and doxorubicin on the redox status of tumor and immune cells. Biophysics 2017, 62 (4), 588-594; and in Jahng, Y., Progress in the Studies on Tryptanthrin, an Alkaloid of History. Archives of Pharmacal Research 2013, 36 (5), 517-535.

Tryptanthrin can also be synthetically obtained from indigo, one of the most stable organic dyes, as described in Pinheiro et al, Tryptanthrin From Indigo: Synthesis, Excited State Deactivation Routes and Efficient Singlet Oxygen Sensitization. Dyes and Pigments 2020, 175, 108125 and Brandão et al, I2/NaH/DMF as oxidant trio for the synthesis of tryptanthrin from indigo or isatin. Dyes and Pigments 2020, 173, 107935.

Tryptanthrin shows two reversible waves with cathodic and anodic peaks, indicating two one-electron transfers, as described in Bhattacharjee et al, Analysis of Stereoelectronic Properties, Mechanism of Action and Pharmacophore of Synthetic Indolo[2,1-b]quinazoline-6,12-dione Derivatives in Relation to Antileishmanial Activity Using Quantum Chemical, Cyclic Voltammetry and 3-D-QSAR CATALYST Procedures. Bioorganic & Medicinal Chemistry 2002, 10 (6), 1979-1989; and in Klimovich et al, A comparative assessment of the effects of alkaloid tryptanthrin, rosmarinic acid, and doxorubicin on the redox status of tumor and immune cells. Biophysics 2017, 62 (4), 588-594.

Similar redox properties have also been reported for some azulene and benzo-annulated tryptanthrin derivatives, as presented in Kogawa et al, Synthesis and Photophysical Properties of Azuleno[1′,2′:4,5]pyrrolo[2,1-b]quinazoline-6,14-diones: Azulene Analogs of Tryptanthrin. Tetrahedron 2018, 74, 7018-7029; and in Liang et al, Synthesis of Benzo-Annulated Tryptanthrins and their Biological Properties. Bioorganic & Medicinal Chemistry 2012, 20 (16), 4962-4967. However, trypthantrin is insoluble in water being not applicable for aqueous redox flow batteries.

### Solution to Problem

In the effort to obtain new electrolytes presenting long-term stability for AORFBs, new polar solvents soluble tryptanthrins, namely, tryptanthrin sulfonic acid (TRYP-SO₃H) and tryptanthrin amine (TRYP-NH₂), were synthesized for the first time and further tested with a home-built RFB set-up. Electrochemical measurements at several pH values, with the determination of the kinetic parameters diffusion coefficient (D) and electron transfer rate constant (k0) of TRYP-SO3H and TRYP-NH₂ at different pH values, namely neutral pH, were obtained.

For tryptanthrin sulfonic acid, charge-discharge processes and cell performance were obtained of (i) aqueous organometallic and (ii) all-organic RFB, combining this new water-soluble tryptanthrin as the negative electrolyte (anolyte) with (i) potassium ferrocyanide and (ii) 4,5-dihydroxy-1,3-benzenedisulfonic acid disodium salt monohydrate (BQDS) as the positive electrolytes (catholytes) at neutral pH.

### Advantageous Effects of Invention

The water-soluble trypthantrin derivatives of the present invention, as well as their salts, when used as soluble electrolytes for aqueous organometallic and all-organic redox flow batteries (RFB), provide them long-term stability, namely when working at neutral pH.

Electrochemical measurements show that water soluble trypthantrin derivatives of the present invention display reversible peaks at several pH values, allowing its use as the anolyte together with organometallic or organic water-soluble catholytes in a neutral supporting electrolyte. The single cell tests present in this description show reproducible charge-discharge cycles for both type of catholytes with significant improvement results for the aqueous all-organic RFB, with coulombic (89%), voltaic (75%) and energetic (67%) efficiencies stabilized during 50 working cycles.

### BRIEF DESCRIPTION OF DRAWINGS

The following figures provide preferred embodiments for illustrating the description and should not be seen as limiting the scope of the present disclosure.

FIG. 1 illustrates a general procedure for the synthesis of compounds of formula (I).

FIG. 2 illustrates a general procedure for the synthesis of compounds of formula (II).

FIG. 3 illustrates cyclic voltammograms of 2.0 mM of TRYP-SO₃H in: (a) pH=0 (1.0 M H₂SO₄) solution, (b) pH=7 (1.0 M KCl) solution and (c) pH=13 (1.0 M NaOH) solution, with saturated N₂, υ=50 mV s⁻¹.

FIG. 4 illustrates cyclic voltammograms of 1.0 mM TRYP-SO₃H (a) and TRYP-NH₂ (b) in 1.0 M KCl solution (left) and 1.0 mM K₄[Fe(CN)₆]3H₂O in 1.0 M KCl solution (right), with saturated N₂, υ=50 mV s⁻¹.

FIG. 5 illustrates cyclic voltammograms of 1.0 mM TRYP-SO₃H in 1.0 M KCl solution (left) and 1.0 mM BQDS in 1.0 M KCl solution (right), with saturated N₂, υ=50 mV s ⁻¹.

FIG. 6 illustrates a cyclic voltammetry scan rate study obtained with saturated N₂ at various scan rates (10 to 100 mV s⁻¹) in 1.0 M KCl solution of electrolyte. a) 1.0 mM TRYP-SO₃H at various scan rates, b) plot of ipa and ipc over the square root of scan rates for 1.0 mM TRYP-SO₃H, c) 1.0 mM K₄[Fe(CN)₆]·3H₂O at various scan rates, d) plot of ipa and ipc over the square root of scan rates for 1.0 mM K₄[Fe(CN)₆]·3H₂O, e) 1.0 mM BQDS at various scan rates, f) plot of ipa and ipc over the square root of scan rates for 1.0 mM BQDS, g) 1.0 mM TRYP-NH2 at various scan rates and h) plot of ipa and ipc over the square root of scan rates for 1.0 mM TRYP-NH₂. In b), d), f) and h) squares and line indicate oxidative reaction and triangles and line indicate reductive reaction.

FIG. 7 illustrates a charge-discharge energy density and charge discharge capacity plots of aqueous organometallic RFB cell test using 5.0 mM of TRYP-SO₃H and 10.0 mM of K₄[Fe(CN)₆]·3H₂O with 1.0 M KCl as supporting electrolyte.

FIG. 8 illustrates the coulombic efficiency, voltaic efficiency, and energetic efficiency plots of neutral pH aqueous organometallic RFB single cells using 5.0 mM of TRYP-SO₃H and 10.0 mM of K₄[Fe(CN)₆]3H₂O in 1.0 M KCl as supporting electrolyte.

FIG. 9 schematic illustration of exemplary RFBs cell tests, wherein aqueous organometallic and all-organic RFB cell using TRYP-SO₃H and K₄[Fe(CN)₆]/BQDS redox couples in neutral pH, and redox reaction mechanisms of TRYP-SO₃H (a), K₄[Fe(CN)₆] (b), and BQDS (c).

FIG. 10 illustrates representative galvanostatic charge-discharge curves of neutral pH aqueous organometallic and all-organic active materials for RFB single cells using 5.0 mM of TRYP-SO₃H in 1.0 M KCl solution as supporting electrolyte measured at the third cycle and at the 50th cycle using (a and b) 10.0 mM of K₄[Fe(CN)₆]·3H₂O as catholyte or (c and d) 5.0 mM of BQDS as catholyte.

FIG. 11 illustrates the charge-discharge energy density and charge-discharge capacity plots of neutral pH aqueous organometallic and all-organic active materials for RFB single cells using 5.0 mM of TRYP-SO₃H in 1.0 M KCl as supporting electrolyte, using (a) 10.0 mM of K₄[Fe(CN)₆]·3H₂O as catholyte or (b) 5.0 mM of BQDS as catholyte.

FIG. 12 illustrates the discharge capacity (black full line) and coulombic efficiency (dashed dot line) vs cycling numbers plots of neutral pH aqueous organometallic and all-organic active materials for RFB single cells using 5.0 mM of TRYP-SO3H in 1.0 M KCl solution as supporting electrolyte using (a) 10.0 mM of K₄[Fe(CN)₆]·3H₂O as catholyte or (b) 5.0 mM of BQDS as catholyte.

FIG. 13 illustrates the obtained coulombic efficiency, voltaic efficiency and energetic efficiency plots of neutral pH aqueous organometallic and all-organic active materials for RFB single cells using 5.0 mM of TRYP-SO₃H in 1.0 M KCl as supporting electrolyte using (a) 10.0 mM of K₄[Fe(CN)₆]·3H₂O as catholyte or (b) 5.0 mM of BQDS as catholyte.

FIG. 14 illustrates the comparison between the catholyte (K₄[Fe(CN)₆]·3H₂O) (a) and anolyte (TRYP-SO₃H) (b) solutions before (black curve) and after (red curve) aqueous organometallic active materials for RFB full cell test using 1.0 M KCl as supporting electrolyte. Solutions with saturated nitrogen (N₂), υ=50 mV s⁻¹.

FIG. 15 illustrates the comparison between the catholyte (BQDS) (a) and anolyte (TRYP-SO₃H) (b) solutions before (black curve) and after (red curve) aqueous organometallic active materials for RFB full cell test using 1.0 M KCl as supporting electrolyte. Solutions with saturated N₂, υ=50 mV s⁻¹.

FIG. 16 illustrates the obtained galvanostatic charge-discharge curves of neutral pH aqueous all-organic active materials for RFB single cell using 0.1 M of TRYP-SO₃H as anolyte and 0.1 M of BQDS as catholyte in 1.0 M KCl solution as supporting electrolyte measured during 50 cycles.

FIG. 17 illustrates the obtained coulombic efficiency, voltaic efficiency, and energetic efficiency plots of neutral pH aqueous all-organic active materials for RFB single cell using 0.1 M of TRYP-SO₃H as anolyte and 0.1 M of BQDS as catholyte in 1.0 M KCl as supporting electrolyte.

FIG. 18 illustrates the obtained charge-discharge energy density and charge-discharge capacity plots of neutral pH aqueous all-organic active materials for RFB single cell using 0.1 M of TRYP-SO₃H and 0.1 M of BQDS as catholyte in 1.0 M KCl as supporting electrolyte.

FIG. 19 illustrates cyclic voltammetry curves comparison between the anolyte (TRYP-SO₃H) before (blue trace) and catholyte (a) (K4[Fe(CN)6]) or (b) BQDS after (red trace) full cell test using 1.0 M KCl as supporting electrolyte. Solutions with saturated N₂, υ=50 mV s⁻¹.

### DESCRIPTION OF EMBODIMENTS

A first aspect of the present invention refers to a trypthantrin derivative of Formula (I)

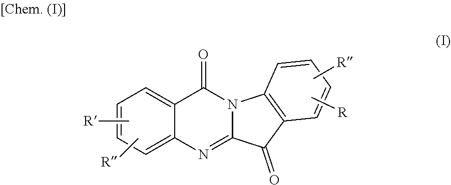

and its salts;

wherein R and R′ are independently H or SO₃H, with the proviso that at least one of R or R′ is SO₃H; and R″ is selected from the group consisting of H, halogen, alkyl, or aryl;

wherein the alkyl group is an alkyl C₁-C₄ linear or branched, preferably selected from the group consisting of methyl, ethyl, n-propyl, n-butyl, iso-butyl or tert-butyl; and

wherein the aryl group is selected from the group consisting of phenyl, hydroxyphenyl, aminophenyl or phenyl sulfonic acid.

In the preferred embodiments, in the trypthantrin derivative of Formula (I), R is SO₃H and R′ is H. In other preferred embodiments, in the trypthantrin derivative of Formula (I), R is H and R′ is SO₃H. In other preferred embodiments, in the trypthantrin derivative of Formula (I), R and R′ are SO₃H.

A second aspect of the present invention refers to a trypthantrin derivative of Formula (II)

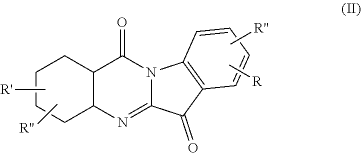

and its salts,

wherein R and R′ are independently H or NH₂, with the proviso that at least one of R or R′ is NH₂; and R″ is selected from the group consisting of H, halogen, alkyl, or aryl;

wherein the alkyl group is an alkyl C₁-C₄ linear or branched, preferably selected from the group consisting of methyl, ethyl, n-propyl, n-butyl, iso-butyl or tert-butyl; and

wherein the aryl group is selected from the group consisting of phenyl, hydroxyphenyl, aminophenyl or phenyl sulfonic acid.

In the preferred embodiments, in the trypthantrin derivative of Formula (II), R is NH₂ and R′ is H.

In other preferred embodiments, in the trypthantrin derivative of Formula (II), R is H and R′ is NH₂. In other preferred embodiments, in the trypthantrin derivative of Formula (II), R and R′ are NH₂.

A third aspect of the present invention refers to a process for the manufacture of the trypthantrin derivative of Formula (I), according to the first aspect, which process comprises a first step of reacting trypthantrin with chlorosulfonic acid; and a subsequent step of hydrolysis to yield said trypthantrin derivative of Formula (I).

A fourth aspect of the present invention refers to a process for the manufacture of the trypthantrin derivative of Formula (II), according to the second aspect, which process comprises a condensation reaction between a compound of Formula (III)

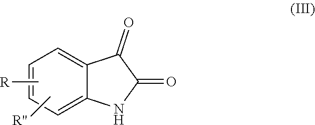

wherein R is independently H or NH₂; and R″ is selected from the group consisting of H, halogen, alkyl, or aryl;

wherein the alkyl group is an alkyl C₁-C₄ linear or branched, preferably selected from the group consisting of methyl, ethyl, n-propyl, n-butyl, iso-butyl or tert-butyl; and

wherein the aryl group is selected from the group consisting of phenyl, hydroxyphenyl, aminophenyl or phenyl sulfonic acid;

with a compound of Formula (IV)

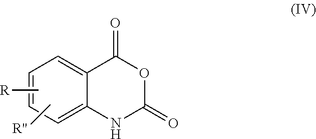

wherein R is independently H or NH₂; and R″ is selected from the group consisting of H, halogen, alkyl, or aryl;

wherein the alkyl group is an alkyl C₁-C₄ linear or branched, preferably selected from the group consisting of methyl, ethyl, n-propyl, n-butyl, iso-butyl or tert-butyl; and

wherein the aryl group is selected from the group consisting of phenyl, hydroxyphenyl, aminophenyl or phenyl sulfonic acid;

to yield said trypthantrin derivative of Formula (II), with the proviso that at least one of R in the compound of Formula (III) or in the compound of Formula (IV) is NH₂.

A fifth aspect of the present invention refers to an anolyte solution for a redox flow battery comprising a polar solvent; and a trypthantrin derivative of Formula (I) of the first aspect, or its salts, as an anolyte material.

The polar solvent is selected from the group consisting of water and solvent:water mixtures, wherein the solvent is miscible in water. Preferably, the solvent miscible in water is selected from the group consisting of ionic liquids, ethanol, glycerol or PEG.

A sixth aspect of the present invention refers to an anolyte solution for a redox flow battery comprising a polar solvent; and a trypthantrin derivative of Formula (II) of the second aspect, or its salts, as an anolyte material.

The polar solvent is selected from the group consisting of water and solvent:water mixtures, wherein the solvent is miscible in water. Preferably, the solvent miscible in water is selected from the group consisting of ionic liquids, ethanol, glycerol or PEG.

In the preferred embodiments, the anolyte solution for a redox flow battery comprising a polar solvent; and a trypthantrin derivative of Formula (I) or its salts further comprises a supporting electrolyte. In the preferred embodiments, the anolyte solution for a redox flow battery comprising a polar solvent; and a trypthantrin derivative of Formula (II) or its salts further comprises a supporting electrolyte.

A seventh aspect of the present invention refers to a redox flow battery comprising the anolyte solution for a redox flow battery comprising a polar solvent; and a trypthantrin derivative of Formula (I) of the first aspect or its salts.

An eighth aspect of the present invention refers to a redox flow battery comprising the anolyte solution for a redox flow battery comprising a polar solvent; and a trypthantrin derivative of Formula (II) of the second aspect or its salts.

In the preferred embodiments, the redox flow battery further comprises a cathode cell comprising a cathode and a catholyte solution; an anode cell comprising an anode and the anolyte solution; and an ion exchange membrane disposed between the cathode cell and the anode cell. In the most preferred embodiments, the catholyte solution comprises a water soluble organometallic catholyte or a water soluble organic catholyte. In the most preferred embodiments, the working pH of the redox flow battery is neutral.

A ninth aspect of the present invention refers to a use of the redox flow battery of the seventh aspect or the eighty aspects in energy storage.

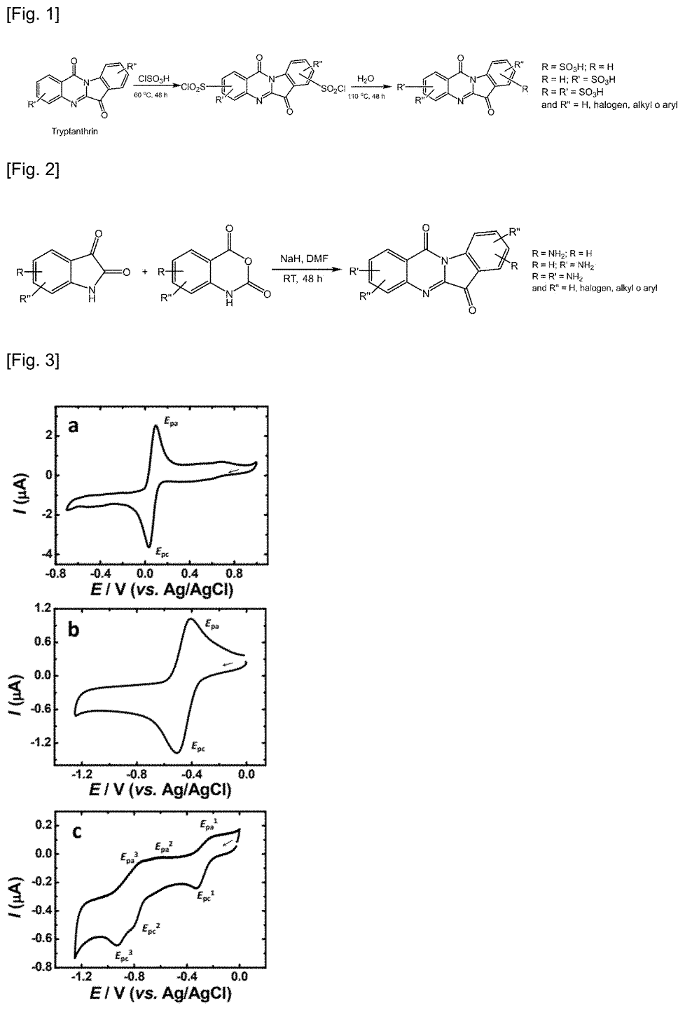

Aiming the preparation of the water-soluble tryptanthrin derivative of Formula (I), a similar synthetic approach to the one reported by Pereira et al. (in Pereira, R. C., Pineiro, M., Galvão, A. M. & Seixas de Melo, J. S. Thioindigo and sulfonated thioindigo derivatives as solvent polarity dependent fluorescent on-off systems. Dyes Pigments 158, 259-266 (2018)) was used. The chlorosulfonic derivatives of TRYP were prepared through electrophilic aromatic substitution with neat chlorosulfonic acid at 60° C. during 48 h under vigorous stirring and nitrogen atmosphere; subsequent hydrolysis of these in water at 110° C. for 48 h lead to the sulfonated (sulfonic acid) compounds of formula (I), as it is illustrated in FIG. 1. The ¹H NMR spectrum showed seven hydrogen atoms, indicating that mono-substitution of tryptanthrin, tryptanthrin sulfonic acid is obtained. These results were confirmed by high-performance liquid chromatography with a diode-array detector (HPLC-DAD) analysis. The infrared (IR) spectrum shows bands at 775, 1200, 1220 and 1350 cm⁻¹ characteristic of hydrated sulfonic acid groups, according to the IR spectrum table by frequency range described in https://www.sigmaaldrich.com/technical-documents/articles/biology/ir-spectrum-table.html.

Trypthantrin bearing amine groups were obtained through a well-established synthetic approach, consisting in the condensation of anthranilic acid derivatives, in particular isatoic anhydride, with isatin in the presence of a base, as it is described in Kawakami et al, Antibacterial and Antifungal Activities of Tryptanthrin Derivatives. Transactions of the Materials Research Society of Japan 2011, 36 (4), 603-606; in Tucker, A. M.; Grundt, P., The Chemistry of Tryptanthrin and Its Derivatives. ARKIVOC 2012, 546-569; and in Li et al, Exploiting 1,2,3-Triazolium Ionic Liquids for Synthesis of Tryptanthrin and Chemoselective Extraction of Copper(II) Ions and Histidine-Containing Peptides. Molecules 2016, 21 (10), 1355. The compounds were dissolved in N,N-dimethylformamide (DMF) with sodium hydride (NaH) and stirred 48 h at room temperature, as illustrated in FIG. 2.

Considering that there are only a few studies describing the redox properties of TRYP (such as in

Klimovich, A. A., Popov, A. M., Krivoshapko, O. N., Shtoda, Y. P. & Tsybulsky, A. V. A comparative assessment of the effects of alkaloid tryptanthrin, rosmarinic acid, and doxorubicin on the redox status of tumor and immune cells. Biophysics 62, 588-594 (2017) and in Jahng, Y. Progress in the studies on tryptanthrin, an alkaloid of history. Arch. Pharm. Res. 36, 517-535 (2013)), the electrochemical behavior of the compounds was initially performed by measuring cyclic voltammetry (CV) of TRYP at different pH values. Cyclic voltammograms (CVs) are presented in FIG. 3 and the relevant electrochemical data including the oxidation (Epa) and reduction (Epc) potentials are summarized in table 1. The CVs obtained showed the occurrence of one reversible reduction peak at 0.034 V and one reversible oxidation peak at 0.097 V at pH=0. At pH=7 similar behavior was also displayed with one cathodic peak at −0.507 V and one anodic peak at −0.406 V. The CV experiment at pH=13 displays three peaks in the anodic region and three peaks in the cathodic region, indicating that the oxidation and reduction processes are reversible. From the obtained results it is visible that TRYP-SO₃H presents high reversible redox behavior, a mandatory condition for its use in aqueous redox flow batteries.

TABLE 1
Electrochemical data including the oxidation (Epa)
and reduction (Epc) potentials for TRYP-SO₃H in
different pH of the supporting electrolyte at T = 293 K

| pH | Oxidation | Reduction |
|---|---|---|
| 0 | Epa = 0.097 V | Epc = −0.034 V |
| 7 | Epa = 0.406 V | Epc = −0.507 V |
| 13 | Epa¹ = 0.225 V | Epc¹ = −0.325 V |
| | Epa² = 0.738 V | Epc² = −0.802 V |
| | Epa³ = 1.178 V | Epc³ = −0.930 V |

Epa¹, Epa² and Epa² = oxidation potential for the first, second and third peak in the voltammogram;
Epc¹, Epc² and Epc² = reduction potential for the first, second and third peak in the voltammogram.

To evaluate the viability of the redox couples consisting on TRYP-SO₃H/K₄[Fe(CN)₆] and TRYP-SO₃H/BQDS (4,5-dihydroxy-1,3-benzenedisulfonic acid disodium salt monohydrate) as active species at neutral pH in the aqueous organometallic RFBs cell, data was obtained from CVs.

Potassium ferrocyanide was chosen as catholyte due to the strong coordination of cyanide ions to the iron center, which makes the standard [Fe(CN)₆]₄-/[Fe(CN)₆]₃-redox couple highly stable and nontoxic. In addition, this redox couple also showed ultra-stable cycling performance at neutral conditions, thus being more suitable for application in aqueous RFBs.

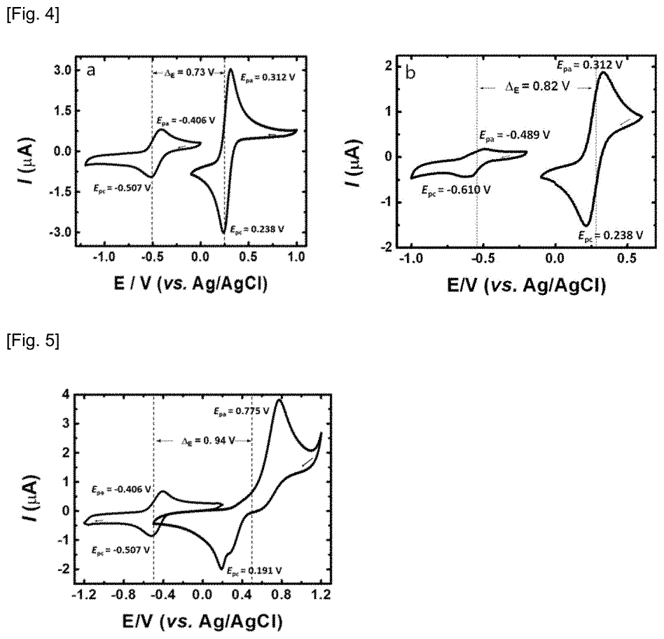

The feasibility of the redox couples TRYP-SO₃H/ferrocyanide and TRYP-NH_{2/}ferrocyanide as active species for neutral pH aqueous organometallic RFBs cells are illustrated in FIGS. 4a and 4b. For TRYP-SO₃H/ferrocyanide, the cell voltage of the redox reaction of K₄[Fe(CN)₆] and TRYP-SO₃H vs. Ag/AgCl is, at neutral pH, +0.27 V and −0.46 V respectively, giving a cell potential of 0.73 V for the TRYP-SO₃H/K₄[Fe(CN)₆] redox couple. For TRYP-NH_{2/}ferrocyanide, the cell voltage of the redox reaction of K₄[Fe(CN)₆] and TRYP-NH₂ vs. Ag/AgCl is, at neutral pH, +0.27 V and −0.45 V respectively, giving a cell potential of 0.82 V for the TRYP-NH₂/K₄[Fe(CN)₆] redox couple.

BQDS is an aromatic organic compound that belongs to the family of quinones and in recent years has been used as the positive active material in aqueous RFBs. Due to a relatively high electrode potential (0.76 V) and high solubility in sulfuric acid (0.65 M in 1.0 M H2SO4), most of the reported studies with BQDS are in acidic medium. Its high solubility in KCl (1.28 M in 1.0 M KCl) and high electrode potential (0.94 V in KCl) demonstrates that BQDS can be viable as positive active material at neutral pH.

Therefore, the feasibility of the redox couple tryptanthrin sulfonic acid/BQDS as active species for neutral pH aqueous all-organic RFBs cell are illustrated in FIG. 5. When the all-organic redox couple TRYP-SO₃H/BQDS is used the cell voltage of BQDS vs. Ag/AgCl is 0.48 V, enhancing the positive shift in the redox potential and achieving a higher cell potential (0.94 V) when compared with TRYP-SO₃H/K₄[Fe(CN)₆] redox couple.

To further verify the correlation between the reaction rate of the catholyte/anolyte pair with the performance and stability of the RFBs, the kinetic parameters, diffusion coefficient (D), and electron transfer rate constant (k0), were quantified.

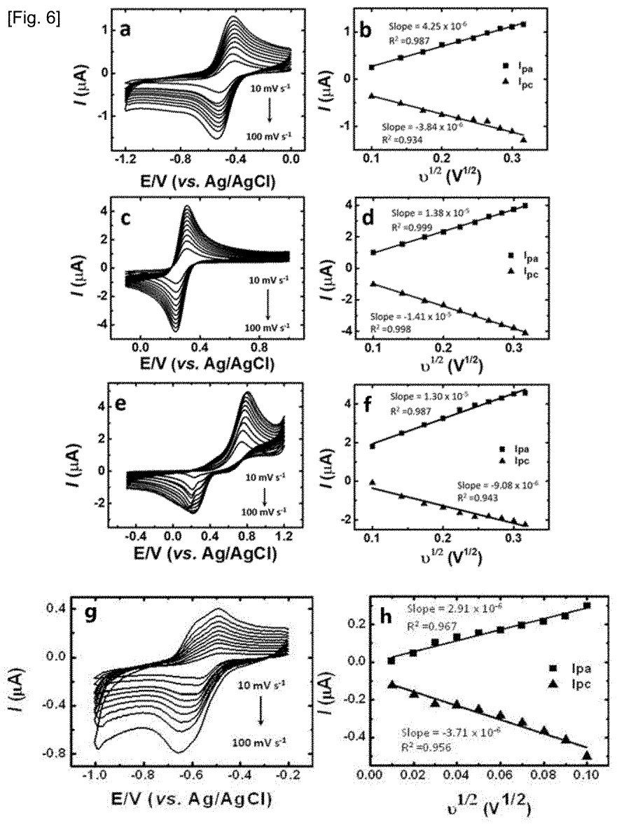

Electrochemical kinetics studies were conducted by CV measurements and Nicholson analysis. To measure the diffusion coefficients of the TRYP-SO3H and TRYP-NH₂ redox couples with K₄[Fe(CN)₆] and BQDS, CV experiments together with the plot of the peak current Ip as function of the square root of the scan rate v½ (FIG. 6) to obtain the Randles-Sevcik equation parameters were performed. The Randles-Sevcik equation, described in Elgrishi et al, A Practical Beginner's Guide to Cyclic Voltammetry. Journal of Chemical Education 2018, 95 (2), 197-206, was used to calculate the diffusion coefficient (D in cm² s⁻¹). A linear fit (ip=slope x v½) yields the slope of the cathodic and anodic peaks from which the D was obtained.

The electron transfer rate constant (k0) of K₄[Fe(CN)₆]/TRYP-SO₃ redox couple was estimated by using the Nicholson method using the D values previously obtained, as described in Nicholson, R. S., Theory and Application of Cyclic Voltammetry for Measurement of Electrode Reaction Kinetics. Analytical Chemistry 1965, 37 (11), 1351-1355. The D and k0 values for K₄[Fe(CN)₆] were (6.63×10⁻⁶ cm s⁻¹ and 1.24×10⁻² cm s⁻¹).

The electron transfer rate constant (k0) of BQDS/TRYP-SO₃ redox couple and TRYP-NH₂ was estimated by using the Nicholson method using the D values previously obtained, as described in Nicholson, R. S., Theory and Application of Cyclic Voltammetry for Measurement of Electrode Reaction Kinetics. Analytical Chemistry 1965, 37 (11), 1351-1355. D and k0 values (5.38×10⁻⁷ cm s⁻¹ and 2.71×10⁻⁴ cm s⁻¹) obtained using BQDS (1.0 mM) were close to those obtained for TRYP-SO³H (6.98×10⁻⁸ cm s⁻¹) and 3.62×10⁻⁴ cm s⁻¹) and the D and k0 values for TRYP-NH₂ was (4.63×10⁻¹ cm s⁻¹ and 2.64×10⁻¹ cm s⁻¹).

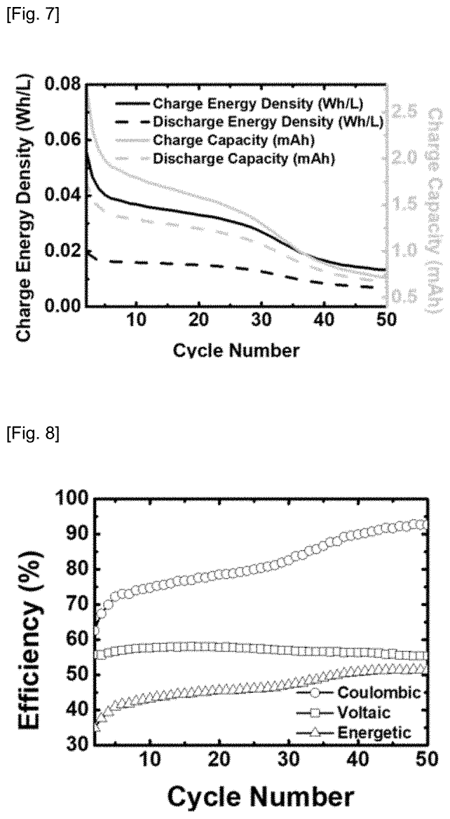

The average discharge energy density and average discharge capacity of the organometallic cell (TRYP-SO₃H/K₄[Fe(CN)₆]) was 0.014 Wh L⁻¹ and 1.17 mAh, respectively (FIG. 7).

The coulombic, voltaic, and energetic efficiencies of the aqueous organometallic flow cell vs. the number of cycles are shown in FIG. 8. For the neutral pH aqueous organometallic RFB the values found for coulombic, voltaic, and energetic efficiencies were 80%, 57% and 46%, respectively.

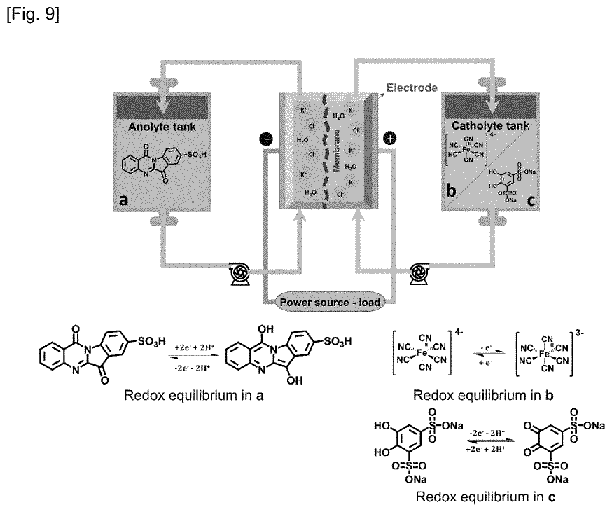

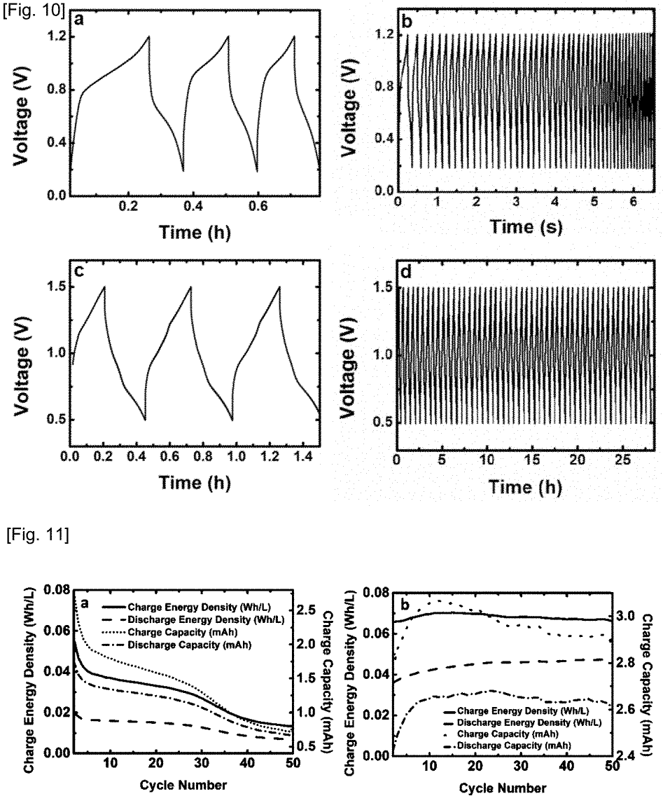

Two single cells were assembled to evaluate the properties of the TRYP-SO₃H/K₄[Fe(CN)]₆ and TRYP-SO₃H/BQDS redox couples for aqueous organometallic and all-organic active materials for RFB working at neutral pH, as shown in exemplary FIG. 9. Representative galvanostatic charge-discharge curves for three cycles and fifty complete cycles of the flow cell obtained for the TRYP-SO₃H/K₄[Fe(CN)₆] and TRYP-SO₃H/BQDS couples with a current density applied of 5.2 and 2.6 mA cm⁻², respectively, are shown in FIG. 10. The cells can be charged and discharged within the selected potential window (cut-off potential set between 0.2 and 1.2 V for TRYP-SO₃H/K₄[Fe(CN)]₆ and between 0.5 and 1.5 V for TRYP-SO₃H/BQDS) with reproducible cycles. The charge-discharge profiles for the two redox couples are slightly different. Indeed, whereas with the TRYP-SO₃H/K₄[Fe(CN)₆ redox couple the interval period between charging and discharging is longer for the first cycle than it is for the others (FIG. 10a), with the TRYP- SO₃H/BQDS redox couple all charge-discharge cycle are identical (FIG. 10c) and stable from the first cycle till the end of the cell test of ˜29 h (50 cycles). It is also noticeable that the charging and discharging time using the all-organic active materials is four times longer than with the organometallic active materials (FIGS. 10b and 10d, respectively).

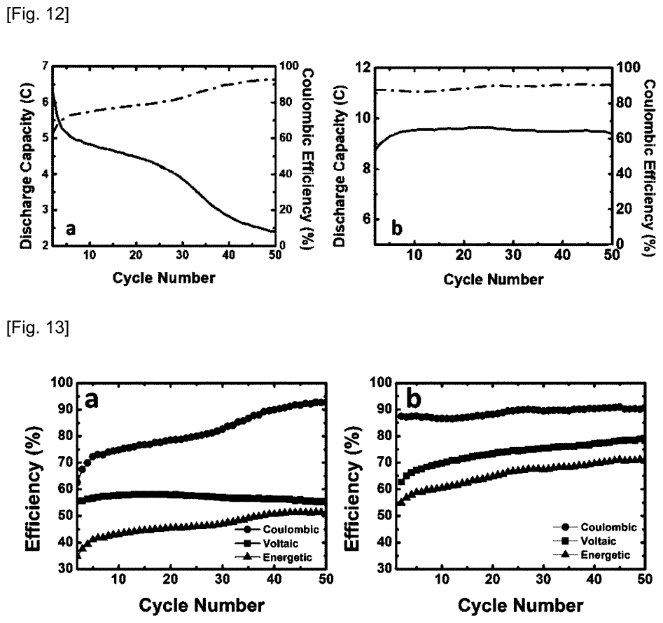

The average discharge energy density and average discharge capacity of the of the organometallic active materials cell were 0.014 Wh L⁻¹ and 1.17 mAh, respectively, while that of the aqueous all-organic TRYP-SO3H/BQDS redox couple is highest, with values of 0.046 Wh L⁻¹ and 2.65 mAh, respectively (FIGS. 11a and 11b). Long-time capacity stability is a vital characteristic for aqueous RFBs. Indeed, while for the organometallic active materials cell (FIG. 11a) during fifty complete cycles (˜7 h) there is a decrease in the charge and discharge energy density and capacity, with capacity retention falling to 38% of its original value (6.309-2.409 C) over fifty cycles (FIG. 12a). In general, non-capacity-related coulombic efficiency loss mainly arises from electrolyte side reactions. In the all-organic active materials cell (FIG. 11b) the values steadily increase until ˜10 cycles and further stabilizes to a total number of fifty cycles in the all-organic active materials cell. Stable capacity retention was observed with more than 98% total capacity retention after forty cycles (FIG. 12b).

The coulombic, voltaic, and energetic efficiencies of the aqueous organometallic and all-organic flow cells vs. the number of cycles are shown in FIG. 13. Coulombic and voltage efficiencies are the properties that better evaluate the performance of RFBs. For the RFB working with neutral pH aqueous organometallic active materials (FIG. 13a) the values found for coulombic, voltaic, and energetic efficiencies were 80%, 57%, and 46%, respectively. The neutral pH aqueous all-organic cell, displayed an interesting cycling performance with 89% coulombic efficiency, 75% of voltaic efficiency and 67% of energy efficiency.

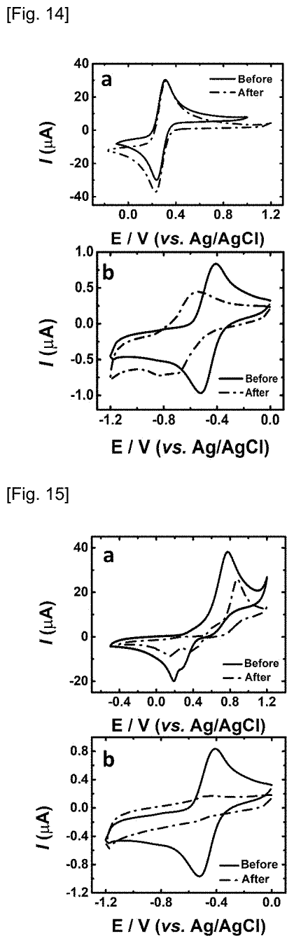

In order to quantify the electrochemical stability of the active materials and to evaluate the possibility of crossover or chemical degradation of the aqueous organometallic and all-organic active materials for RFB during cycling, electrochemical impedance spectra were measured before and after charge-discharge cycles, see FIGS. 14 and 15. From the voltaic efficiency, it is possible to assess losses through electrolyte crossover.

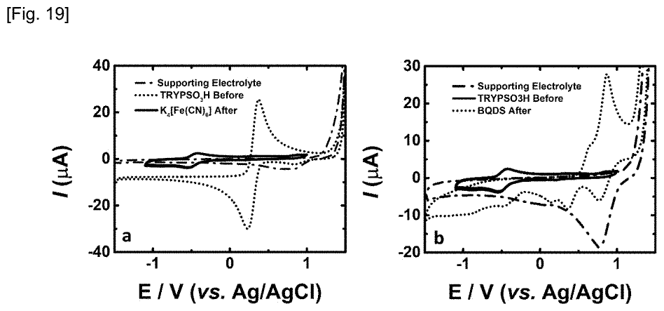

During the cell test, there were no vestiges of crossover through the membrane. After the cell measurement, the CV measurement performed to the catholyte (K₄[Fe(CN)₆]) (FIG. 14a) does not differ from the initial one (FIG. 4) and no peaks in the CV of the K₄[Fe(CN)₆] electrolyte in the range of the CV of TRYP-SO₃H (FIG. 19a) could be observed. According to FIG. 14b, and after full cell tests, a change in the voltammogram profile of TRYP-So₃H is seen, with the peak maxima shifting to more negative potentials together with new anodic and cathodic peaks.

These results may help to explain the changes observed in the values of the charge-discharge cycles with time, as well as the observed battery capacity loss, which is likely related to some chemical modification of TRYP-SO₃H after 50 cycles. From FIG. 15a, the cyclic voltammetry curves of the BQDS electrolyte, obtained before and after the charge-discharge studies, are shown to be also different. After finalizing the experiment with the RFB, the BQDS active material showed an additional redox peak.

The instability of BQDS has already been reported in an acidic medium, where this compound can undergo a Michael addition reaction with water leading to trihydroxybenzene derivatives (as reported by Hoober-Burkhardt, L. et al. A new Michael-reaction-resistant benzoquinone for aqueous organic redox flow batteries. J. Electrochem. Soc. 164, A600-A607 (2017), Lai, Y. Y. et al. Stable low-cost organic dye anolyte for aqueous organic redox flow battery. ACS Appl. Energy Mater. 3, 2290-2295 (2020), Rubio-Garcia, J., Kucernak, A., Parra-Puerto, A., Liu, R. & Chakrabarti, B. Hydrogen/functionalized benzoquinone for a high-performance regenerative fuel cell as a potential large-scale energy storage platform. J. Mater. Chem. A 8, 3933-3941 (2020) and Yang, B. et al. High-performance aqueous organic flow battery with quinone-based redox couples at both electrodes. J. Electrochem. Soc. 163, A1442-A1449 (2016)). This reaction may also explain the obtained results at neutral pH values which is further reflected in the capacity decrease. According to FIG. 15b, before cycling, the TRYP-SO₃H electrolyte has a current intensity of the reduction and oxidation peak much higher than after fifty charge-discharge cycles indicating loss of reversibility.

Crossover through the membrane was also investigated. After the charge-discharge experiment ended, no peaks in the cyclic voltammogram of the BQDS electrolyte, in the range of the TRYP-SO₃H peaks, could be observed (FIG. 19b), indicating the absence of crossover of the TRYP-SO₃H electrolyte. Another possibility for the capacity fading is the leakage from the cell stack system. After cell cycling, both cells were disassembled and no coloration was found on the gaskets, indicating the absence of electrolyte leakage.

Both cell tests showed the absence of crossover of the redox couple through the membrane (the electrolyte volume in the two reservoirs was kept the same after 50 complete cycles). It is known that despite the many advantages displayed by AORFBs, one of the main disadvantages is their lower energy density. The energy density of the TRYP-SO₃H/BQDS all-organic redox couple (0.046 Wh L⁻¹) is comparable with previous studies of neutral pH AORFBs, as described in Luo et al, Unprecedented Capacity and Stability of Ammonium Ferrocyanide Catholyte in pH Neutral Aqueous Redox Flow Batteries. Joule 2019, 3 (1), 149-163; in Hu et al, Long-Cycling Aqueous Organic Redox Flow Battery (AORFB) toward Sustainable and Safe Energy Storage. Journal of the American Chemical Society 2017, 139 (3), 1207-1214; in Hu et al, A pH-Neutral, Metal-Free Aqueous Organic Redox Flow Battery Employing an Ammonium Anthraquinone Anolyte. Angewandte Chemie International Edition 2019, 58 (46), 16629-16636; and in Hu et al, Boosting the energy efficiency and power performance of neutral aqueous organic redox flow batteries. Journal of Materials Chemistry A 2017, 5 (42), 22137-22145.

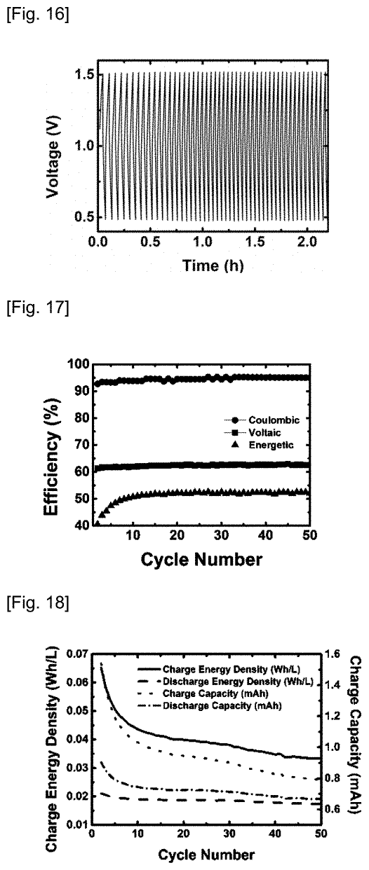

In order to study the effect of the concentration of the all-organic active materials in the stability and performance of the RFB, the concentration of the redox pair was increased to 0.1 M, a concentration close to the solubility limit of TRYP-SO3H (0.12 M in 1.0 M KCl). The data is presented in FIGS. 16 and 17 and indicates the following: (i) with the augment on the concentration of TRYP-SO₃H and BQDS, the active materials remain well dissolved and operated well during 50 cycles (FIG. 16); (ii) as the concentration of TRYP-SO3H and BQDS increased, the coulombic efficiency reaches 95% (FIG. 17); (iii) with 0.1 M of active materials, the discharge energy density and capacity values are, however, lower (FIG. 18) when compared with the highest values displayed by other systems working at neutral pH values such as the ones disclosed in Luo, J. et al. Unprecedented capacity and stability of ammonium ferrocyanide catholyte in pH neutral aqueous redox flow batteries. Joule 3, 149-163 (2019), Hu, B., DeBruler, C., Rhodes, Z. & Liu, T. L. Long-cycling aqueous organic redox flow battery (AORFB) toward sustainable and safe energy storage. J. Am. Chem. Soc. 139, 1207-1214 (2017), Hu, B., Luo, J., Hu, M., Yuan, B. & Liu, T. L. A pH-neutral, metal-free aqueous organic redox flow battery employing an ammonium anthraquinone anolyte. Angew. Chem. Int. Ed. 58, 16629-16636 (2019), Hu, B., Seefeldt, C., DeBruler, C. & Liu, T. L. Boosting the energy efficiency and power performance of neutral aqueous organic redox flow batteries. J. Mater. Chem. A 5, 22137-22145 (2017) and Sanchez-Dliez, E. et al. Redox flow batteries: status and perspective towards sustainable stationary energy storage. J. Power Sources 481, 228804 (2021).

### EXAMPLES

As example of compounds with general formula (I). 300 mg of tryptanthrin (1.2 mmol) was placed in a bottom flask under nitrogen and heated with a paraffin bath at 60° C. Keeping an inert atmosphere, chlorosulfonic acid (3 mL, 45 mmol) was added and the mixture was stirred for 48 h. The solution was left to cool in ice and neutralized with a saturated solution of sodium bicarbonate (NaHCO₃). The garnet solid obtained was filtrated and washed with water and the solid was dried at 45° C. overnight to yield 173 mg of a dark green solid. NMR analysis of the reaction crude showed that the dark green solid consisted in a mixture of, tryptanthrin sulfonyl chloride and some unreacted tryptanthrin. The dark green mixture (100 mg) was suspended in 50 mL of water and further heated at 110° C. for 48 h until a green solution was obtained. After cooling to room temperature, the solution was filtrated to remove a trace of non-dissolved tryptanthrin and the solvent was evaporated under vacuum. The green solid obtained was dried at 45° C. for 24 h to yield 73 mg of a mixture of two isomers, 6,12-dioxo-6,12-dihydroindolo[2,1-b]cquinazoline-8-sulfonic acid, tryptanthrin 8-sulfonic acid (TRYP-SO₃H) and 6,12-dioxo-6,12-dihydroindolo[2,1-b]quinazoline-2-sulfonic acid, tryptanthrin 2-sulfonic acid (TRYP-SO₃H) in an 85:15 ratio. 1H NMR (CDCl₃, 400 MHz,), δ 8.69 (d, J=1.6 Hz, 1H), 8.44 (ddd, J=8.0 Hz, J=1.6 Hz, J=0.4 Hz, 1H), 8.05 (ddd, J=8.4 Hz, J=1.2 Hz, J=0.8 Hz, 1H), 7.87 (m, 2H), 7.70 (dt, J=8.4 Hz, J=1.2 Hz, 1H), 7.40 (dd, J=8.0 Hz, J=1.6 Hz, 1H) ppm. IR (KBr pellets) wavenumber (cm⁻¹): 775 cm⁻¹, 1200 cm⁻¹, 1220 cm−1, 1350 cm⁻¹, 1430 cm⁻¹, 1590 cm⁻¹, 1680 cm⁻¹, 1730 cm⁻¹. HPLC-DAD: Stationary phase Purospher™ STAR RP-18 endcapped (5 μm). Eluent: potassium acetate (pH=3)(A) and MeCN:H₂O (50:50)(B) (from 100:0 to 30:70(A/B v/v)); 5% increment of B each min for 12 min; 5% increment of B each 2 min until 22 min and 30:70 (NB v/v) ratio for 12 min. Flow rate of 0.8 mL/min in the first 9 min and then 0.4 mL/min until 35 min. L-2455 Diode Array Detector (400 nm). Percentage of total chromatogram integration at retention time 29.49 min and 31.89 min of 72% and 14% respectively.

As example of compounds with general formula (II). A solution of isatin (500 mg, 3.4 mmol) in 10 mL of DMF was added over a 15-minute period to NaH (82 mg, 3.4 mmol) with stirring. To the resulting red liquid, 5-aminoisatoic anhydride (660 mg, 3.7 mmol) in 10 mL of DMF was added with ice cooling over a 30-minute period. The reaction mixture was stirred 48 h at room temperature and then quenched with 10 mL of methanol. The resulting mixture was diluted with 50 mL of chloroform and washed once with water. The aqueous layer was extracted three times with chloroform and the combined organic layers were concentrated under reduce pressure. Crystallization from acetone afforded the pure 2-aminoindolo[2,1-b]quinazoline-6,12-dione, 2-aminotryptanthrin (TRYP-NH₂) as a dark brown solid (410 mg, 82% yield). 1H NMR (CDCl3, 400 MHz), 6 8.62 (d, J=8.1 Hz, 1H), 7.89 (d, J=7.5 Hz, 1H), 7.83 (d, J=8.7 Hz, 1H), 7.78-7.72 (m, 1H), 7.58 (d, J=2.7 Hz, 1H), 7.40 (t, J=7.6 Hz, 1H), 7.11 (dd, J=2.7 Hz, J=8.7 Hz, 1H), 4.33 (s, 2H) ppm. ¹³C NMR (CDCl₃, 101 MHz), δ 193.22, 168.36, 155.50, 148.84, 138.60, 137.70, 132.57, 126.93, 125.08, 122.19, 118.00, 117.99, 117.92, 111.60, 110.12 ppm. HRMS (ESI-TOF-MS): m/z [M+1]+=264.0770 calculated for C₁₅H₁₀N₃O₂; found: 264.0768.

Cyclic voltammetry (CV) experiments were carried out using an Autolab potentiostat/galvanostat PGSTAT204 running with NOVA 2.1 software and a three-electrode system in a one-compartment electrochemical cell of capacity 10 mL. A glassy carbon electrode (GCE) (d=3 mm) was the working electrode, glassy carbon (GC) (d=1.6 mm) wires the counter electrode and Ag/AgCl (3.0 M KCl) the reference electrode. The GCE was polished with appropriate polishing pads using first aluminium oxide with particle size 0.3 μm and then aluminium oxide particle with size 0.075 μm (polish in a FIG. 7 motion) before each electrochemical experiment. After polishing, the electrode was rinsed thoroughly with Milli-Q water and the electrode was sonicate in a container with Milli-Q water and ethanol (50:50 v/v) for 5 minutes. Following this mechanical treatment, the GCE was placed in buffer supporting electrolyte and differential pulse voltammograms were recorded until a steady state baseline voltammogram was obtained. This procedure ensured very reproducible experimental results.

To evaluate open circuit voltage (OCV) between TRYP-SO₃H and K₃[Fe(CN)₆] at different pH values, the solutions were prepared at a concentration of 2.0 mM and degassed with nitrogen for 10 min prior to analysis. Depending on the pH experiments different electrolytes were used, for pH=0 (1.0 M H₂SO₄) solution, for pH=7 (1.0 M KCl) solution and pH =13 (1.0 M NaOH) solution. Potassium ferricyanide (K₃[Fe(CN)₆]·3H₂O) (1.0 mM solution) with 1.0 M of the proper electrolyte in 10 mL of Milli-Q water was used as standard and also degassed with nitrogen for 10 minutes prior to analysis. Lastly, to measure the OCV for BQDS, 1.0 mM was dissolved into 1.0 M KCl solution. An atmosphere of nitrogen was maintained during the voltammetric experiments, and the samples were run at a scan rate of 50 mV s⁻¹.

For the determination of the kinetic parameters, such as diffusion coefficient (D) and electron transfer rate constant (k0), a solution of TRYP-SO₃H (1.0 mM) was dissolved in 10 mL of Milli-Q water with 1.0 M KCl as the supporting electrolyte. First, a solution of 1.0 mM of K₄[Fe(CN)₆] with 1.0 M of KCl in 10 mL of Milli-Q water was used as catholyte. Second, the catholyte was prepared by dissolving 1.0 mM of BQDS into 10 mL of 1.0 M KCl solution. All the solutions were degassed with nitrogen for 10 min prior to analysis. An atmosphere of nitrogen was maintained during the voltammetric experiments, and the samples were run at a scan rate of 10-100 mV s⁻¹ range. All the CV experiments were carried out at T=293 K.

The flow cell for the AORFBs was assembled with two steel end frame plates and two copper current collectors, held in place using two carbon electrolyte chambers. Graphite foil was used to form a flexible interconnect to the copper endplate. Ethylene propylene diene monomer (EPDM) rubber gaskets were positioned on top of the carbon plate and the carbon felt electrodes (Alfa Aesar, 3.18 mm) were positioned within the gaskets. A piece of Nafion™ perfluorinated membrane (Aldrich, nafion™ 115) was sandwiched between carbon felts and the battery was compressed using tie-bolts. Each carbon chamber was connected with an electrolyte reservoir using a piece of Viton type tube. The electrolyte reservoirs were 100 mL glass containers. The active area of the cell was 4 cm2. A Master™ L/S™ peristaltic pump (Cole-Parmer, Easy-load II, Model 77202-60) was used to press sections of Masterflex tubing to circulate the electrolytes through the electrodes at a flow rate of 30 mL min⁻¹. Both reservoirs were purged with nitrogen to remove O₂ for 30 minutes and an atmosphere of nitrogen was maintained during the cell cycling. The flow cell was galvanostatically charged/discharged at room temperature and measurements were carried out with a current density applied of 20 mA using 0.2 and 1.2 V cut-off potentials in the first test (TRYP-SO₃H/K₄[Fe(CN)₆]) and a current density applied of 10 mA using 0.5 and 1.5 V cut-off potentials in the second test (TRYP-SO₃H/BQDS). The charge-discharge curves were recorded using an Autolab potentiostat/galvanostat PGSTAT204 running with NOVA 2.1 software. The negative electrolyte was prepared by dissolving TRYP-SO₃H (5.0 mM) in 50 mL of Milli-Q water with 1.0 M KCl as the supporting electrolyte. First, a solution of 10.0 mM of K₄[Fe(CN)₆] with 1.0 M of KCl in 50 mL of Milli-Q water was used as positive electrolyte. Second, the catholyte was prepared by dissolving 5.0 mM of BQDS into 50 mL of 1.0 M KCl solution. All the solutions were degassed with nitrogen for 30 minutes prior to analysis and an atmosphere of nitrogen was maintained during cell cycling. All experiments were carried out at room temperature (T=293 K).

For organic impurities removal the nafion perfluorinated membrane was initially emerged in Milli-Q water at 80° C. for 15 minutes and then put into 5% hydrogen peroxide solution (H202) for 30 minutes. In the next step in order to cleanse the metallic impurities, the membrane was put into a 0.05 M KCl solution for one hour (after 30 minutes the KCl solution was changed). In the last step the membrane was put into Milli-Q water for one hour, changing the water every 15 minutes. After pre-treatment, the membrane was placed in Milli-Q water to avoid further contaminations.

A piece of carbon felt was heated at 400° C. for 24 hours in a muffle furnace Vulcan 3-550. Then, the temperature of the muffle furnace was lowered to room temperature and the carbon felt was removed and properly stored until further use.

### CITATION LIST

Citation List follows:

### Non-Patent Literature

NPL1: Winsberg, J.; Stolze, C.; Muench, S.; Liedl, F.; Hager, M. D.; Schubert, U.S., TEMPO/Phenazine Combi-Molecule: A Redox-Active Material for Symmetric Aqueous Redox-Flow Batteries. ACS Energy Letters 2016, 1 (5), 976-980

NPL2: Huang, C.-Y.; Bonasera, A.; Hristov, L.; Garmshausen, Y.; Schmidt, B. M.; Jacquemin, D.; Hecht, S., N,N′-Disubstituted Indigos as Readily Available Red-Light Photoswitches with Tunable Thermal Half-Lives. Journal of the American Chemical Society 2017, 139 (42), 15205-15211

NPL3: Lin, K.; Chen, Q.; Gerhardt, M. R.; Tong, L.; Kim, S. B.; Eisenach, L.; Valle, A. W.; Hardee, D.; Gordon, R. G.; Aziz, M. J.; Marshak, M. P., Alkaline Quinone Flow Battery. Science 2015, 349 (6255), 1529-1532

NPL4: Wang, W.; Luo, Q.; Li, B.; Wei, X.; Li, L.; Yang, Z., Recent Progress in Redox Flow Battery Research and Development. Advanced Functional Materials 2013, 23 (8), 970-986

NPL5: Chen, R., Redox Flow Batteries for Energy Storage: Recent Advances in Using Organic Active Materials. Current Opinion in Electrochemistry 2020, 21, 40-45

NPL6: Liu, Y.; Lu, S.; Chen, S.; Wang, H.; Zhang, J.; Xiang, Y., A Sustainable Redox Flow Battery with Alizarin-Based Aqueous Organic Electrolyte. ACS Applied Energy Materials 2019, 2 (4), 2469-2474

NPL7: Singh, V.; Kim, S.; Kang, J.; Byon, H. R., Aqueous Organic Redox Flow Batteries. Nano Research 2019, 12, 1988-2001

NPL8: Liu, W.; Lu, W.; Zhang, H.; Li, X., Aqueous Flow Batteries: Research and Development. Chemistry—A European Journal 2019, 25 (7), 1649-1664

NPL9: Hoober-Burkhardt, L.; Krishnamoorthy, S.; Yang, B.; Murali, A.; Nirmalchandar, A.; Prakash, G. K. S.; Narayanan, S. R., A New Michael-Reaction-Resistant Benzoquinone for Aqueous Organic Redox Flow Batteries. Journal of The Electrochemical Society 2017, 164 (4), A600-A607

NPL10: Winsberg, J.; Hagemann, T.; Janoschka, T.; Hager, M. D.; Schubert, U. S., Redox-Flow Batteries: From Metals to Organic Redox-Active Materials. Angewandte Chemie International Edition 2017, 56 (3), 686-711

NPL11: Wei, X.; Pan, W.; Duan, W.; Hollas, A.; Yang, Z.; Li, B.; Nie, Z.; Liu, J.; Reed, D.; Wang, W.; Sprenkle, V., Materials and Systems for Organic Redox Flow Batteries: Status and Challenges. ACS Energy Letters 2017, 2 (9), 2187-2204

NPL12: Lv, Y.; Liu, Y.; Feng, T.; Zhang, J.; Lu, S.; Wang, H.; Xiang, Y., Structure Reorganization-Controlled Electron Transfer of Bipyridine Derivatives as Organic Redox Couples. Journal of Materials Chemistry A 2019, 7 (47), 27016-27022

NPL13: Luo, J.; Hu, B.; Debruler, C.; Bi, Y.; Zhao, Y.; Yuan, B.; Hu, M.; Wu, W.; Liu, T. L., Unprecedented Capacity and Stability of Ammonium Ferrocyanide Catholyte in pH Neutral Aqueous Redox Flow Batteries. Joule 2019, 3 (1), 149-163

NPL14: Hu, B.; DeBruler, C.; Rhodes, Z.; Liu, T. L., Long-Cycling Aqueous Organic Redox Flow Battery (AORFB) toward Sustainable and Safe Energy Storage. Journal of the American Chemical Society 2017, 139 (3), 1207-1214

NPL15: DeBruler, C.; Hu, B.; Moss, J.; Liu, X.; Luo, J.; Sun, Y.; Liu, T. L., Designer Two-Electron Storage Viologen Anolyte Materials for Neutral Aqueous Organic Redox Flow Batteries. Chem 2017, 3 (6), 961-978

NPL16: Hofmann, J. D.; Schroder, D., Which Parameter is Governing for Aqueous Redox Flow Batteries with Organic Active Material? Chemie Ingenieur Technik 2019, 91 (6), 786-794

NPL17: Hollas, A.; Wei, X.; Murugesan, V.; Nie, Z.; Li, B.; Reed, D.; Liu, J.; Sprenkle, V.; Wang, W., A Biomimetic High-Capacity Phenazine-Based Anolyte for Aqueous Organic Redox Flow Batteries. Nature Energy 2018, 3 (6), 508-514

NPL18: Lee, W.; Permatasari, A.; Kwon, B. W.; Kwon, Y., Performance evaluation of aqueous organic redox flow battery using anthraquinone-2,7-disulfonic acid disodium salt and potassium iodide redox couple. Chemical Engineering Journal 2019, 358, 1438-1445

NPL19: Lee, W.; Permatasari, A.; Kwon, Y., Neutral pH Aqueous Redox Flow Batteries Using an Anthraquinone-Ferrocyanide Redox Couple. Journal of Materials Chemistry C 2020

NPL20: Permatasari, A.; Lee, W.; Kwon, Y., Performance Improvement by Novel Activation

Process Effect of Aqueous Organic Redox Flow Battery using Tiron and Anthraquinone-2,7-Disulfonic Acid Redox Couple. Chemical Engineering Journal 2020, 383, 123085

NPL21: Gerken, J. B.; Anson, C. W.; Preger, Y.; Symons, P. G.; Genders, J. D.; Qiu, Y.; Li, W.; Root, T. W.; Stahl, S. S., Comparison of Quinone-Based Catholytes for Aqueous Redox Flow Batteries and Demonstration of Long-Term Stability with Tetrasubstituted Quinones. Advanced Energy Materials 2020, 10 (20), 2000340

NPL22: Hu, B.; Luo, J.; Hu, M.; Yuan, B.; Liu, T. L., A pH-Neutral, Metal-Free Aqueous Organic Redox Flow Battery Employing an Ammonium Anthraquinone Anolyte. Angewandte Chemie International Edition 2019, 58 (46), 16629-16636

NPL23: Beh, E. S.; De Porcellinis, D.; Gracia, R. L.; Xia, K. T.; Gordon, R. G.; Aziz, M. J., A Neutral pH Aqueous Organic-Organometallic Redox Flow Battery with Extremely High-Capacity Retention. ACS Energy Letters 2017, 2 (3), 639-644

NPL24: Hu, B.; Seefeldt, C.; DeBruler, C.; Liu, T. L., Boosting the energy efficiency and power performance of neutral aqueous organic redox flow batteries. Journal of Materials Chemistry A 2017, 5 (42), 22137-22145

NPL25: Chen, H.; Cong, G.; Lu, Y.-C., Recent Progress in Organic Redox Flow Batteries: Active Materials, Electrolytes and Membranes. Journal of Energy Chemistry 2018, 27 (5), 1304-1325

NPL26: Huskinson, B.; Marshak, M. P.; Suh, C.; Er, S.; Gerhardt, M. R.; Galvin, C. J.; Chen, X.; Aspuru-Guzik, A.; Gordon, R. G.; Aziz, M. J., A Metal-Free Organic-Inorganic Aqueous Flow Battery. Nature 2014, 505, 195

NPL27: Lin, K.; Gómez-Bombarelli, R.; Beh, E. S.; Tong, L.; Chen, Q.; Valle, A.; Aspuru-Guzik, A.; Aziz, M. J.; Gordon, R. G., A Redox-Flow Battery with an Alloxazine-Based Organic Electrolyte. Nature Energy 2016, 1 (9), 16102

NPL28: Kaur, R.; Manjal, S. K.; Rawal, R. K.; Kumar, K., Recent Synthetic and Medicinal Perspectives of Tryptanthrin. Bioorganic & Medicinal Chemistry 2017, 25 (17), 4533-4552

NPL29: Deryabin, P. I.; Mosk0vkina, T. V.; Shevchenk0, L. S.; Kalinovskii, A. I., Synthesis and antimicrobial activity of tryptanthrin adducts with ketones. Russian Journal of Organic Chemistry 2017, 53 (3), 418-422

NPL30: Novak, M. J.; Clayton Baum, J.; Buhrow, J. W.; Olson, J. A., Scanning Tunneling Microscopy of Indolo[2,1-b]quinazolin-6,12-dione (tryptanthrin) on HOPG: Evidence of Adsorption-Induced Stereoisomerization. Surface Science 600 2006, (20), L269-L273

NPL31: Bhattacharjee, A. K.; Skanchy, D. J.; Jennings, B.; Hudson, T. H.; Brendle, J. J.; Werbovetz, K. A., Analysis of Stereoelectronic Properties, Mechanism of Action and Pharmacophore of Synthetic Indolo[2,1-b]quinazoline-6,12-dione Derivatives in Relation to Antileishmanial Activity Using Quantum Chemical, Cyclic Voltammetry and 3-D-QSAR CATALYST Procedures. Bioorganic & Medicinal Chemistry 2002, 10 (6), 1979-1989

NPL32: Kawakami, J.; Matsushima, N.; Ogawa, Y.; Kakinami, H.; Nakane, A.; Kitahara, H.; Nagaki, M.; Ito, S., Antibacterial and Antifungal Activities of Tryptanthrin Derivatives. Transactions of the Materials Research Society of Japan 2011, 36 (4), 603-606

NPL33: Filatov, A. S.; Knyazev, N. A.; Shmak0v, S. V.; Bogdanov, A. A.; Ryazantsev, M. N.; Shtyrov, A. A.; Starova, G. L.; Molchanov, A. P.; Larina, A. G.; Boitsov, V. M.; Stepak0v, A. V., Concise Synthesis of Tryptanthrin Spiro Analogues with In Vitro Antitumor Activity Based on One-Pot, Three-Component 1,3-Dipolar Cycloaddition of Azomethine Ylides to Cyclopropenes. Synthesis 2019, 51 (03), 713-729

NPL34: Amara, R.; Awad, H.; Chaker, D.; Bentabed-Ababsa, G.; Lassagne, F.; Erb, W.; Chevallier, F.; Roisned, T.; Dorcet, V.; Fajloun, Z.; Vidal, J.; Mongin, F., Conversion of Isatins to Tryptanthrins, Heterocycles Endowed with a Myriad of Bioactivities. European Journal of Organic Chemistry 2019, (31-32), 5302-5312

NPL35: Klimovich, A. A.; Popov, A. M.; Krivoshapk0, O. N.; Shtoda, Y. P.; Tsybulsky, A. V., A comparative assessment of the effects of alkaloid tryptanthrin, rosmarinic acid, and doxorubicin on the redox status of tumor and immune cells. Biophysics 2017, 62 (4), 588-594

NPL36: Jahng, Y., Progress in the Studies on Tryptanthrin, an Alkaloid of History. Archives of Pharmacal Research 2013, 36 (5), 517-535

NPL37: Pinheiro, D.; Pineiro, M.; Pina, J.; Brenda), P.; Galva), A. M.; Seixas de Melo, J. S., Tryptanthrin From Indigo: Synthesis, Excited State Deactivation Routes and Efficient Singlet Oxygen Sensitization. Dyes and Pigments 2020, 175, 108125

NPL38: Seixas de Melo, J. S., The Molecules of Colour and Art. Molecules With History and Modern Applications. In Photochemistry, Albini, A.; Prodi, S., Eds. RSC: London, 2020; Vol. 47, pp 196-216

NPL39: Pina, J.; Sarmento, D.; Accoto, M.; Gentili, P. L.; Vaccaro, L.; Galva), A.; Seixas de Melo, J. S., Excited-State Proton Transfer in Indigo. The Journal of Physical Chemistry B 2017, 121 (10), 2308-2318

NPL40: Seixas de Melo, J. S.; Burrows, H. D.; Serpa, C.; Arnaut, L. G., The Triplet State of Indigo. Angewandte Chemie International Edition 2007, 46 (12), 2094-2096

NPL41: K0gawa, C.; Fujiwara, A.; Sekiguchi, R.; Shoji, T.; Kawakami, J.; Okazaki, M.; Ito, S., Synthesis and Photophysical Properties of Azuleno[1′,2′:4,5]pyrrolo[2,1-b]quinazoline-6,14-diones: Azulene Analogs of Tryptanthrin. Tetrahedron 2018, 74, 7018-7029

NPL42: Liang, J. L.; Park, S.-E.; Kwon, Y.; Jahng, Y., Synthesis of Benzo-Annulated Tryptanthrins and their Biological Properties. Bioorganic & Medicinal Chemistry 2012, 20 (16), 4962-4967

NPL43: Tucker, A. M.; Grundt, P., The Chemistry of Tryptanthrin and Its Derivatives. ARKIVOC 2012, 546-569

NPL44: https://www.sigmaaldrich.com/technical-documents/articles/biology/ir-spectrum-table.html

NPL45: Li, H.-Y.; Chen, C.-Y.; Cheng, H.-T.; Chu, Y.-H., Exploiting 1,2,3-Triazolium Ionic Liquids for Synthesis of Tryptanthrin and Chemoselective Extraction of Copper(II) Ions and Histidine-Containing Peptides. Molecules 2016, 21 (10), 1355

NPL46: Elgrishi, N.; Rountree, K. J.; McCarthy, B. D.; Rountree, E. S.; Eisenhart, T. T.; Dempsey, J. L., A Practical Beginner's Guide to Cyclic Voltammetry. Journal of Chemical Education 2018, 95 (2), 197-206

NPL47: Nicholson, R. S., Theory and Application of Cyclic Voltammetry for Measurement of Electrode Reaction Kinetics. Analytical Chemistry 1965, 37 (11), 1351-1355

NPL48: Alotto, P. et al. Redox flow batteries for the storage of renewable energy: a review. Renew. Sustain. Energy Rev. 29, 325-335 (2014)

NPL49: Carretero-Gonzalez, J. et al. Highly water-soluble three-redox state organic dyes as bifunctional analytes. Energy Environ. Sci. 9, 3521-3530 (2016)

NPL50: Khalid, A. & Awad, M. Chapter 5—Redox—Principles and Advanced Applications. (IntchOpen, 2017); Huskinson, B. et al. A metal-free organic-inorganic aqueous flow battery. Nature 505, 195 (2014)

NPL51: Shigematsu, T. Redox flow battery for energy storage. Sei Tech. Rev. 73, 4-13 (2011)

NPL52: Noack, J. et al. The chemistry of redox-flow batteries. Angew. Chem. Int. Ed. 54, 9776-9809 (2015)

NPL53: Brandão et al, I2/NaH/DMF as oxidant trio for the synthesis of tryptanthrin from indigo or isatin. Dyes and Pigments 2020, 173, 107935.

NPL54: Pereira, R. C., Pineiro, M., Galvõ, A. M. & Seixas de Melo, J. S. Thioindigo and sulfonated thioindigo derivatives as solvent polarity dependent fluorescent on-off systems. Dyes Pigments 158, 259-266 (2018)

1.-22. (canceled)

23. A trypthantrin derivative of Formula (I) 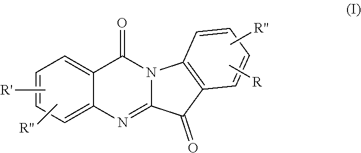
and its salts;
wherein
R and R′ are independently H or SO3H, with the proviso that at least one of R or R′ is SO₃H; and
R″ is selected from the group consisting of H, halogen, alkyl, or aryl;
wherein the alkyl group is an alkyl C₁-C₄ linear or branched, preferably selected from the group consisting of methyl, ethyl, n-propyl, n-butyl, iso-butyl or tert-butyl; and
wherein the aryl group is selected from the group consisting of phenyl, hydroxyphenyl, aminophenyl or phenyl sulfonic acid.

24. The trypthantrin derivative of claim 23, wherein R is SO3H and R′ is H.

25. The trypthantrin derivative of claim 23, wherein R is H and R′ is SO₃H.

26. The trypthantrin derivative of claim 23, wherein R and R′ are SO3H.

27. The trypthantrin derivative of claim 23 selected from the group consisting of:
6,12-dioxo-6,12-dihydroindolo[2,1-b]quinazoline-8-sulfonic acid, tryptanthrin 8-sulfonic acid;
6,12-dioxo-6,12-dihydroindolo[2,1-b]quinazoline-2-sulfonic acid, tryptanthrin 2-sulfonic acid.

28. A trypthantrin derivative of Formula (II) 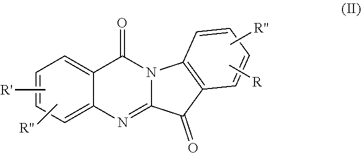
and its salts;
wherein
R and R′ are independently H or NH₂, with the proviso that at least one of R or R′ is NH₂; and
R″ is selected from the group consisting of H, halogen, alkyl, or aryl;
wherein the alkyl group is an alkyl C₁-C₄ linear or branched, preferably selected from the group consisting of methyl, ethyl, n-propyl, n-butyl, iso-butyl or tert-butyl; and
wherein the aryl group is selected from the group consisting of phenyl, hydroxyphenyl, aminophenyl or phenyl sulfonic acid.

29. The trypthantrin derivative of claim 28, wherein R is NH2 and R′ is H.

30. The trypthantrin derivative of claim 28, wherein R is H and R′ is NH2.

31. The trypthantrin derivative of claim 28, wherein R and R′ are NH2.

32. The trypthantrin derivative of claim 28, that is 2-aminoindolo[2,1-b]quinazoline-6,12-dione, 2-aminotryptanthrin.

33. A process for the manufacture of the trypthantrin derivative of Formula (I), as defined in claim 23, which process comprises a first step of reacting trypthantrin with chlorosulfonic acid; and a subsequent step of hydrolysis to yield said trypthantrin derivative of Formula (I).

34. A process for the manufacture of the trypthantrin derivative of Formula (II), as defined in claim 28, which process comprises a condensation reaction between a compound of Formula (III) 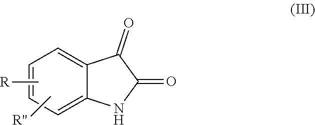
wherein
R is independently H or NH₂; and
R″ is selected from the group consisting of H, halogen, alkyl or aryl;
wherein the alkyl group is an alkyl C₁-C₄ linear or branched, preferably selected from the group consisting of methyl, ethyl, n-propyl, n-butyl, iso-butyl or tert-butyl; and
wherein the aryl group is selected from the group consisting of phenyl, hydroxyphenyl, aminophenyl or phenyl sulfonic acid;
with a compound of Formula (IV) 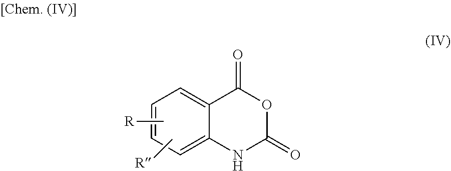
wherein
R is independently H or NH₂; and
R″ is selected from the group consisting of H, halogen, alkyl, or aryl;
wherein the alkyl group is an alkyl C₁-C₄ linear or branched, preferably selected from the group consisting of methyl, ethyl, n-propyl, n-butyl, iso-butyl or tert-butyl; and
wherein the aryl group is selected from the group consisting of phenyl, hydroxyphenyl, aminophenyl or phenyl sulfonic acid,
to yield said trypthantrin derivative of Formula (II), with the proviso that at least one of R in the compound of Formula (III) or in the compound of Formula (IV) is NH₂.

35. An anolyte solution for a redox flow battery comprising a polar solvent; and a trypthantrin derivative of Formula (I), as defined in claim 23, as an anolyte material.

36. The anolyte solution for a redox flow battery of claim 35, wherein the polar solvent is selected from the group consisting of water and solvent:water mixtures, wherein the solvent is a solvent miscible in water selected from the group consisting of ionic liquids, ethanol, glycerol or PEG.

37. The anolyte solution for a redox flow battery of claims 35 further comprising a supporting electrolyte.

38. An anolyte solution for a redox flow battery comprising a polar solvent; and a trypthantrin derivative of Formula (II), as defined in claim 28, as an anolyte material.

39. The anolyte solution for a redox flow battery of claim 38, wherein the polar solvent is selected from the group consisting of water and solvent:water mixtures, wherein the solvent is a solvent miscible in water selected from the group consisting of ionic liquids, ethanol, glycerol or PEG.

40. The anolyte solution for a redox flow battery of claim 38 further comprising a supporting electrolyte.

41. A redox flow battery comprising the anolyte solution, as defined in claim 35.

42. The redox flow battery of claim 41, further comprising a cathode cell comprising a cathode and a catholyte solution; an anode cell comprising an anode and the anolyte solution; and an ion exchange membrane disposed between the cathode cell and the anode cell, wherein the catholyte solution comprises a water soluble organometallic catholyte or a water soluble organic catholyte and the working pH is neutral.

43. A redox flow battery comprising the anolyte solution, as defined in claim 38.

44. The redox flow battery claim 43, further comprising a cathode cell comprising a cathode and a catholyte solution; an anode cell comprising an anode and the anolyte solution; and an ion exchange membrane disposed between the cathode cell and the anode cell, wherein the catholyte solution comprises a water soluble organometallic catholyte or a water soluble organic catholyte and the working pH is neutral.

45. Use of the redox flow battery, as defined in claim 41, in energy storage.

46. Use of the redox flow battery, as defined in claim 43, in energy storage.
